(19) United States
(12) Patent Application Publication
(10) Pub. No.: US 2023/0094184 A1
(43) Pub. Date: Mar. 30, 2023

(54) SYSTEM AND METHOD TO HOLD TRIALING SYSTEM

(71) Applicant: Medtronic, Inc., Minneapolis, MN (US)

(72) Inventors: Michael J. BAADE, Otsego, MN (US); Steven T. DEININGER, Plymouth, MN (US); Jeff John CLAYTON, Zimmerman, MN (US)

(21) Appl. No.: 17/855,007

(22) Filed: Jun. 30, 2022

Related U.S. Application Data
(60) Provisional application No. 63/247,376, filed on Sep. 23, 2021.

(51) Int. Cl. A61N 1/372

(52) U.S. Cl. CPC A61N 1/37241

(57) ABSTRACT

Disclosed is a medical assembly, according to various embodiments, for providing a therapy to a subject. The assembly is operable to be held relative to a subject for a trial period. The holder may allow for connections and/or passage of connections from the medical device to allow for appropriate or a selected operation of the medical device.

### CROSS REFERENCE TO RELATED APPLICATIONS

This application claims the benefit of U.S. Provisional Application No. 63/247,376, filed on Sep. 23, 2021. The entire disclosure(s) of (each of) the above application(s) is (are) incorporated herein by reference.

### FIELD

The subject application relates to a medical system, and particularly to a trialing system for a stimulation assembly.

### BACKGROUND

This section provides background information related to the present disclosure which is not necessarily prior art.

An implantable device may be designed to provide appropriate stimulation to a selected patient. The implantable device may be designed with selected characteristics, inputs and outputs, and other physical features. Nevertheless, prior to finalization and/or implantation of the implantable device a trialing of various features of the device may be performed.

In trialing the features, an external system may be developed that includes controls, stimulation parameters, and other specifications that are similar to the implantable device. The evaluation system, however, is developed to be positioned external to a subject, thus not requiring the implantation and/or ease of manipulation for testing and evaluation. Various evaluation systems may be made for stimulation systems, such as the Interstim® stimulation system, sold by Medtronic, Inc. The evaluation system may generally be designed for ease of programming and positioning externally to a subject and, therefore, may include various geometries and dimensions inappropriate for implantation.

### SUMMARY

This section provides a general summary of the disclosure, and is not a comprehensive disclosure of its full scope or all of its features.

A stimulation device is provided to stimulate a subject, such as a human patient, for various therapies. Therapies may include pain relief, neurostimulation, muscle control, or other appropriate therapies. The stimulation is provided with a stimulation device that is positioned within a patient semi- or permanently and/or provided external to the patient for a temporary trial period. The stimulation device may be referred to as a neurostimulator (NS). The stimulation device designed for internal placement, such as implantation, may be referred to as an implantable neurostimulation (INS) system or device. The device may be positioned externally as a trialing or external neurostimulation (ENS) system.

A pouch may be carried and/or adhered to a subject for a selected period of time, such as a trialing period of time. The trial may include a selected amount of time, such as three days, seven days, or any appropriate number of days or hours. The pouch may hold a holder that may also be referred to as a stabilizer, housing, or the like. The holder is generally held in the pouch or by the pouch relative to the subject. As discussed herein, the pouch may include a wearable pouch that is held by a strap or other mechanism and/or may include an adhesive that adheres the pouch to the subject.

The holder is used to hold the implantable neurostimulation device (INS) for example the RestoreSensor® neurostimulator system and/or other appropriate neurostimulator systems that may include the Adaptivestim® stimulation technology and included system. It is understood that any appropriate INS may be used to be held by the holder for a selected period of time, as discussed further herein. The INS may be trialed when in the holder and therefore may be connected with selected stimulation leads to the subject and appropriate grounding leads to the subject. Thus, the INS may be trialed in an external position when positioned with the holder without requiring implantation of the INS. Moreover, trialing may occur with the INS that is the exact same INS to be implanted and/or identical to the INS to be implanted without requiring a separate trialing system.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a schematic environmental view of a holder and pouch, according to various embodiments;

FIG. 2 is an exemplary view of an implantable medical device, such as an implantable neurostimulator (INS), according to various embodiments;

FIG. 3 is a perspective view of a holder and INS, according to various embodiments;

FIG. 4 is a top plane view of the holder of FIG. 1, according to various embodiments;

FIG. 5 is a cross-sectional view, without an INS, of the holder of FIG. 4 taken along line 5-5;

FIG. 6 is a cross-sectional view taken along line 6-6 of FIG. 4;

FIG. 7 is a holder with a positioned INS, according to various embodiments;

FIG. 8 is a top plane view of the holder of FIG. 7 with an INS, according to various embodiments;

FIG. 9 is a cross-sectional view taken along line 9-9 of FIG. 7;

FIG. 10 is a cross-sectional view taken along lines 10-10 of FIG. 7;

FIG. 11 is a perspective view of a holder, according to various embodiments;

FIG. 12 is a cross-sectional view of FIG. 11 taken along lines 12-12;

FIG. 13 is a first perspective view of a holder and a first cover;

FIG. 14 is a second perspective view of a holder and a second cover; and

FIG. 15 is an exploded view of a holder, an INS, a first cover, and a second cover.

Corresponding reference numerals indicate corresponding parts throughout the several views of the drawings.

### DETAILED DESCRIPTION

Example embodiments will now be described more fully with reference to the accompanying drawings.

The subject disclosure relates to various subjects, such as human patients. It is understood, however, that a holder, as discussed further herein, may be used relative to living subjects that are not human subjects and/or non-living subjects. For example, the holder may be used to hold a selected device externally to an airframe while being connected to various internal sensors and/or leads for a selected period of time. Accordingly, it is understood that the holder may be used for human subjects and/or non-human subjects. Further, the holder may be used to hold a stimulation system that is designed to be implanted. The implantable neurostimulation system (INS) may be implanted at a selected time. The INS, however, may position the holder, as discussed further herein, for a trialing period. It is further understood that other implantable systems, such as cardiac pacemakers, defibrillators, and the like may also be positioned in a holder for a selected trialing period if selected.

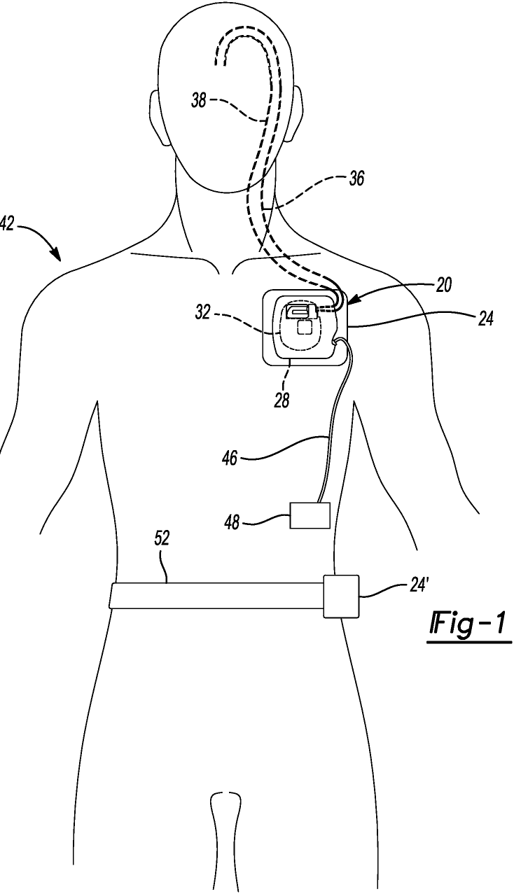

With initial reference to FIG. 1, an external trialing system 20 is illustrated. The external trialing system 20 may include an external pouch 24 and external holder 28, and an implantable stimulator system (INS) 32. Connected to the INS 32 may be one or more leads 36, 38 to provide therapy to a subject 42. In the exemplary system illustrated in FIG. 1, the leads 36, 38 may be deep brain stimulation leads that are positioned within a portion of a brain of a subject 42. Further, a ground lead 46 is connected to the subject 42, such as with an adhesive patch or ground connection 48. The ground lead 46 allows grounding to the subject 42 at a position away from or a distance from the leads 36, 38.

While the trialing system 20 is illustrated as having the pouch 24 adhered to the subject 42, it is understood that the pouch 24 may be positioned at an alternative position relative to the subject 42. For example, an alternative pouch 24ʹ may be worn with a belt 52 relative to the patient 42 and the leads 36, 38, 46 may extend from the pouch 24ʹ rather than the pouch 24. Also, pouches may be held relative to the subject 42 in the appropriate manner, such as with a shoulder or chest strap, or other appropriate holding system. Accordingly, the subject disclosure of the adhesive pouch 24 is merely exemplary of a pouch that may hold a holder 28 relative to the subject 22 for a trial period.

Each of the leads 36, 38, 48 may connect with the INS 32 in an appropriate manner. The leads may then extend from the holder 28 to interconnect with selected portions of the subject 42, such as being implanted into the brain and/or connected to the subject at the ground patch 48. It is understood that the respective leads may be connected to the subject 42 in a manner that substantially mimics and/or is identical to an implanted positioning.

The holder 28 may be provided in various embodiments, including those discussed further herein. Each of the various embodiments may be referenced with the same referencing augmented with one or more primes (ʹ). It is understood, however, that the holder 28 may be provided in any appropriate embodiment and/or incorporated portions or features of more than one embodiment, including those examples as discussed further herein.

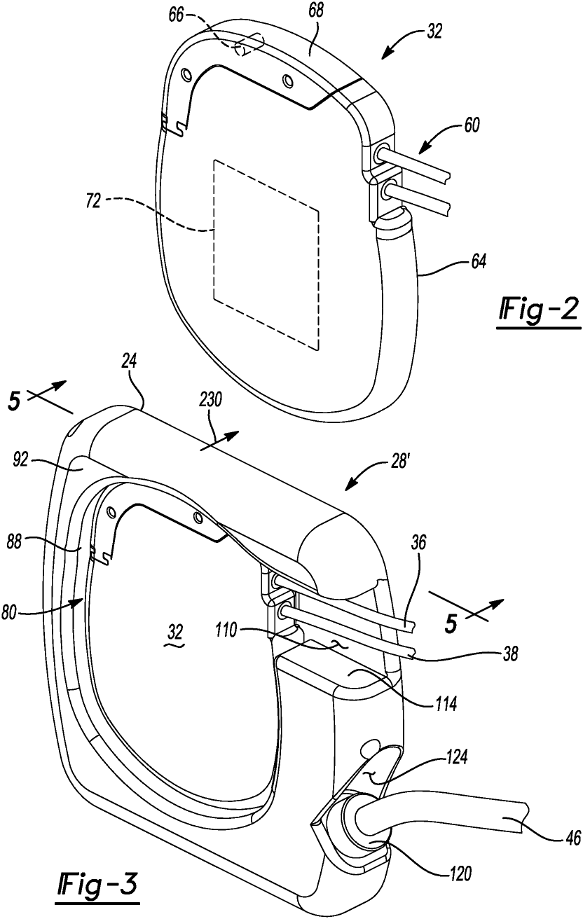

Turning reference to FIG. 2, the INS 32 may include various features, including those discussed herein. The INS 32 may include one or more ports or a port region 60 for connection of the leads 36, 38. Thus, ports in the port region 60 may be for input (e.g., sensing) to the INS 32 and/or output therefrom (e.g., stimulation). It is understood that the port region 60 may include a plurality of ports to accept or connect with any appropriate number of leads, such as two, four, ten, twenty or any appropriate number. The INS 32 generally includes a case or housing 64 that may be formed of a conductive material, such as a metal or a metal alloy. In various embodiments, the case 64 allows for grounding of the INS 32, for a selected operation thereof. The case 64 may be or act as a ground electrode and an electrical connection therewith may provide a ground for the INS 32. Further, the INS 32 may include various features, such as a wireless transmission and/or receiving assembly. The wireless receiving assembly may include an antenna 66 that is covered by a non-conductive or non-interfering material or cover 68. The cover 68 may be a selected polymer or non-conductive material. Therefore, communication with the INS 32 may be wireless, as is generally understood in the art. The INS 32 may house electronic components 72 that may be operated for therapy of the subject 42, such as neurostimulation, muscular stimulation, or the like. The electronic components 72 may be similar to those known in the art to provide appropriate therapies and/or later developed and may provide stimulation through the ports 60 and/or be grounded though the case 64. Thus, the electronic components 72 allow the INS 32 to operate as intended. The INS 32 may be an implantable device that may be identical and/or the exact same device to be implanted in the subject 42 at a selected time. During a trialing phase, such as is prior to approval for general use, planning for a specific patient, or any appropriate trialing, the INS 32 may be positioned within a holder 28.

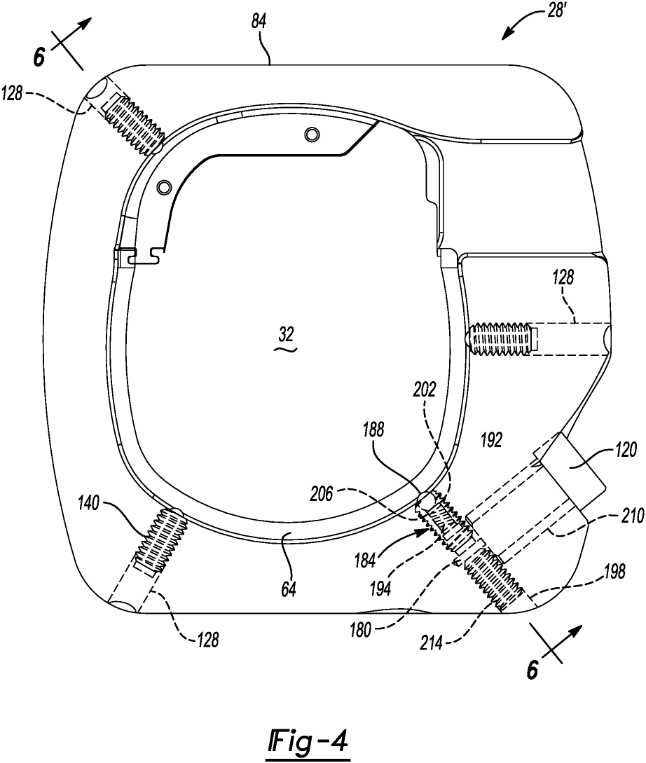

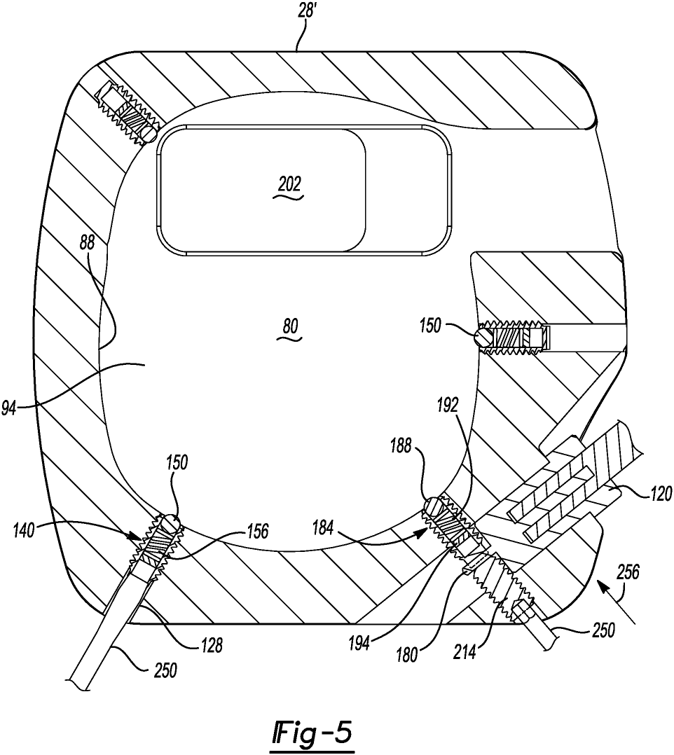

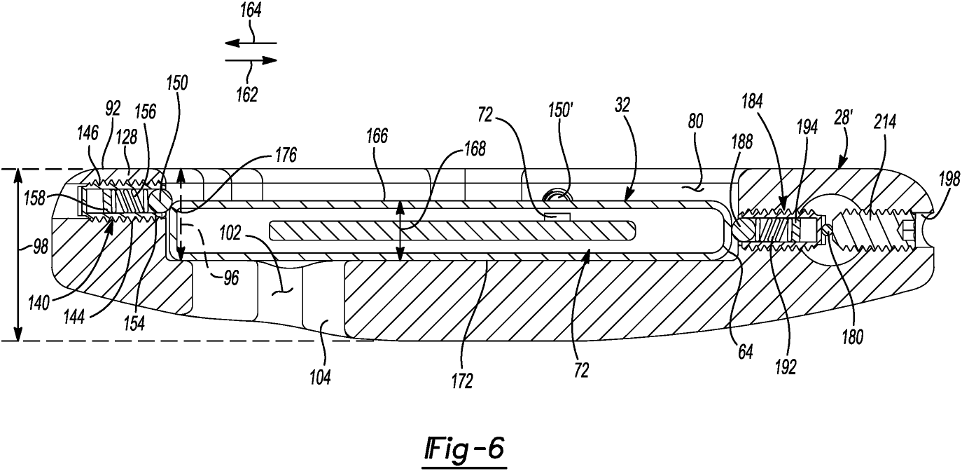

With reference to FIG. 3, FIG. 4, FIG. 5, and FIG. 6, the INS 32 may be positioned in a holder 28ʹ. The INS 32 may be positioned within a well or a depression 80 of the holder 28ʹ. The depression 80 may be formed within an exterior wall 84 of the holder 28ʹ. The depression 80 may be formed by one or more internal walls 88. The internal walls 88 may extend from an upper surface 92 to an internal bottom surface 94. The internal wall 88, therefore, may include a selected dimension 96. The dimension 96 may be any appropriate dimension, such as one that allows for a receipt of the INS 32 to maintain the INS 32 within the depression 80 of the holder 28ʹ, ensure proper contact with the INS 32 within the holder 28ʹ or other appropriate purposes. In various embodiments, for example, the dimension 96 may be about 0.5 mm to about 20 mm, including about 1 mm to about 12 mm.

The dimension 96 may be provided to hold an appropriate INS that may be of a selected size or dimension based upon the selected INS, or other appropriate features. Moreover, the holder 28ʹ may include an overall dimension or a thickness 98 that may be standard for any INS dimension to ensure that it is configured or operable to be received within the pouch 24. Accordingly, a plurality of INS devices may be fit within a single holder exterior dimension to fit within the pouch 24 by forming the depression 80 of the holder 28 to receive and hold the selected INS relative to the holder 28. Also, as discussed herein, selected tolerances of sizes of different INSs 32 may be fit within the depression 80 of the holder 28 due to the selected holding portions.

The holder 28 may be formed of selected materials that may generally be selected to be non-electrically conductive. For example, however, the holder 28 may be formed of an electrically conductive material that is coated in selected portions with a non- electrically conducting material to insulate the INS 32 relative to the holder 28. In various embodiments, however, the holder 28 may be formed of a polymer and/or co-polymer material. In various embodiments, for example, the holder 28 may be formed of a polyether ether ketone (PEEK), Liquid Silicone Rubber, polysulfone, polypropylene, polyurethane, polyetherimide, Liquid Crystal Polymer, or other appropriate polymer materials.

The holder 28 may be formed of the selected material in a selected process. For example, the holder 28 may be formed by molding, 3D printing, casting, machining from a solid block, or the like. Any appropriate manufacturing process, however, may be used to effectively form the holder 28 as one skilled in the art would understand. Further, the holder 28 may be formed to include various features, including those discussed further herein, to hold the INS 32 and allow for trialing of the INS 32.

The holder 28ʹ, for example, may include one or more ports or passages 102. The passage 102 may be defined by one or more internal walls 104 through the external surface or portion of the holder 28ʹ. The portal 102 may allow for engagement of the INS 32, for example, to disengage the INS 32 from the holder 28ʹ. The depression 80, otherwise, of the holder 28ʹ may include various shapes and configurations to engage the hold the INS 32 within the holder 28ʹ.

In various embodiments, for example, the holder 28ʹ further includes a lead passage or side port 110 that may be formed or defined by one or more internal side walls 114 to allow passage of the leads 36, 38 from the INS 32 and away from the holder 28ʹ to the subject 42. The passage 110 allows the leads 36, 38 to extend from the INS 32 in a substantially unobstructed manner for ease of manipulation and positioning by the user, such as the surgeon, physician, or the like.

The ground lead 46 may engage a coupling 120 that allows for grounding of the INS 32 relative to the subject 42. A passage or portal 124 may be formed through the holder 28ʹ to allow for extension of the ground lead 46 away from the holder 28ʹ. As discussed herein, the passage 124 allows for the ground lead 46 to be in electrical connection with the INS 32.

With continuing reference to FIGS. 3-6, and with particular reference to FIGS. 4-6, the holder 28ʹ may define a plurality of holding bores or positioning bores 128 formed through the exterior portion or wall 84. Positioned within each of the holding bores and/or appropriate number of the holding bores 128, may be one or more biasing holding assemblies 140. Each of the biasing holding assemblies 140 may include or define an external thread 144 that engages an internal thread 146 of the holder 28ʹ. It is understood that the holding bores 128 may also include inserts, such as a helical member, that may assist in increasing a holding power of the biasing holding assemblies 140. Alternatively and/or additionally, however, the internal thread 146 may be formed directly into the holder 28ʹ.

Each of the biasing holding assemblies 140 may include a holding portion that may be a plunger ball 150 that is captured within an outer cylindrical member 154 that may define the external thread 144. A biasing member 156 is also provided within the biasing holding assemblies 140, such as with a wall or base portion 158. The biasing member 156 may, therefore, hold or bias the plunger ball toward the INS 32 and/or the depression 80.

Thus, the ball 150 may be biased generally in the direction of arrow 162. The ball 154 then moves in the direction of arrow 164, such as when engaged by the INS 32 as it is being inserted into the depression 80 and/or removed from the depression 80. The holding ball 150 may be depressed into the holding cylinder 154 and then biased into the engaging or holding position when the INS 32 is positioned at a selected position relative to the holder assembly 140.

For example, as illustrated in FIG. 6, the INS 32 includes an outer surface or portion 166. The outer surface 166 has a dimension 168 from a bottom or internal or second surface 172. The dimension 168 may assist in defining a positioning of the holding ball 150 within the holder 28ʹ. For example, as illustrated in FIG. 6, the holding assembly 140, including the holding ball 150, may be positioned to engage the upper surface 166 and/or an upper radius 176 of the INS 32 when the INS 32 is in the engaged placed in the depression 80. As discussed above, a plurality of the holding assemblies 140 may be positioned in a plurality of the holding bores 128 to hold the INS 32 at different holding points to assist in the maintaining the INS 32 within the holder 28ʹ. The internal dimensions of the depression 80 may, therefore, be substantially close or similar to the external dimensions of the INS 32, without requiring exacting dimensions to ensure that the INS 32 is held within the holder 28ʹv. The holding assemblies 140 may allow for a selected tolerance, such as about 0.01 millimeters (mm) (about 0.0004 inch) to about 25 mm (about 1 inch), including about 0.6 mm (about 0.25 inches) of the dimensions of the depression 80 while ensuring that the INS 32 is held within the holder 28ʹ with an appropriate holding force. For example, the ball 150 may move about 0.01 millimeters (mm) (about 0.0004 inch) to about 25 mm (about 1 inch), including about 0.6 mm (about 0.25 inches) The holding assemblies may apply a holding force such as to resist at least 0.1 Newtons (N) of force on the INS 32 being removed from the holder 28ʹ. The force to remove the INS 32 form the holder 28ʹ may, however, be selected to be about 0.4 N (0.1 pounds) to about 44 N (10 pounds) of force.

The holding assemblies 140 may be any appropriate holding member such as one or more set screws, set screw plunger, and/or compressive material (e.g., elastic material). The materials of the holding assemblies 140 may be selected to work with the materials of the holder 28' and/or the INS 32. For example, the holding ball 50, the biasing member 156, and the cylinder 154 may all be formed of a selected material, such as a stainless steel, including a medical grade stainless steel to ensure compatibility with the INS 32. In various embodiments, the holding assemblies 140 may be electrically conductive. Further, various portions of each of the holding assemblies 140 may be formed of a material that would not mar or damage the INS 32. For example, the holding ball 150 may be formed of a soft material, such as a rigid polymer, coated metal or metal alloy, or other appropriate configuration. Nevertheless the holding assemblies 140 may be provided to hold the INS 32 within the holder 28ʹ.

The holder 28ʹ has interconnected therewith and/or includes the grounding plug 120. The lead 46 engages the grounding plug 120. The grounding plug 120 connects and/or contacts the case 64 of the INS 32. As discussed above, the case 64 may be a ground electrode for the INS 32. Therefore, by the grounding plug 120 connecting to or contacting the case 64, the grounding plug 120 may also be a grounding contact for the INS 32. As discussed above, the grounding lead 46 may be connected to a grounding patch or connection 48 to ground the INS 32 to the subject 42.

The grounding plug 120 may be engaged into the holder 28ʹ in any appropriate manner, such as with an adhesive, a thread, an interference fit, combinations thereof, or the like. The grounding plug 120 includes one or more contact portions, such as a contact wire 180. The contact wire 180 contacts a ground holding assembly 184. The ground holding assembly 184 may be similar to the holding assemblies 140, as discussed above, save that the grounding assembly 184 contacts the case 64 of the INS 32 to ensure an appropriate and constant electrical connection to the case 64 to ground 42. The grounding assembly 64, therefore, may include a grounding ball 188 that may be formed of a conductive material, such as stainless steel, as discussed above. Further, the grounding assembly 184 may include a grounding biasing member, such as a spring 192 that may include a stainless steel spring. Any other appropriate conductive biasing portion may also be used to bias the grounding ball 188 against the case 64. As discussed above, the biasing member 192 may be held within the grounding assembly 184 with an appropriate wall or holding surface 194.

The grounding assembly 184 may be positioned within a grounding bore or passage 198 formed within the holding member 28’. The grounding passage 198 may be substantially similar to the holding passages 128, as discussed above. The grounding passage 198, may be molded, milled, or the like formed in the holder 28’. The grounding assembly 184, therefore, may include an external thread 202 that engages an internal thread 206 within the bore 198. As also discussed above, a threaded insert or coil portion, such as a helical insert, may also be provided within the passage 198 to assist in ensuring appropriate holding force of the grounding assembly 184 within the passage 198.

During assembly of the holder 28', the grounding assembly 184 may be positioned within the grounding bore 188. The grounding plug 120 may then be assembled within a grounding plug passage 210 formed in the holder 28' such that the contact 180 extends into and/or through the grounding passage 198. The contact 180 may then be contacted, such as compressed or held, relative to the grounding assembly 184 with a set screw 214. The set screw 214 may also be threaded into the grounding bore 198 to engage and contact the contact 180 and compress or holding against the grounding assembly 184. Therefore, the set screw 214 may be assembled into the grounding bore 198 in an appropriate manner or time, such as after the assembly or positioning of the INS 32 within the holder 28ʹ.

During use, therefore, the INS 32 may be positioned within the holder 28' by a selected user prior to or concurrent with positioning in the pouch 24. The user may move the INS 32 into the depression 80, such as moving the INS 32 generally in the direction of arrow 230 (FIG. 3). The holding assemblies 140 may allow for elastic movement of the holding members 150 against the biasing portions or members 156 within the holding assemblies 140. Once the INS 32 is positioned in an appropriate portion or position within the depression 80, the holding members 150 may be biased into the holding position by the biasing members 156 to then hold the INS 32 within the holder 28’. A user may engage one or more of the holding assemblies 140 with a tool, such as a hex driver 250 to move the holding assembly 140 within the holding bores 128 to ensure an appropriate holding force of the INS 32 within the holder 28’ and/or to ease positioning of the INS 32 within the holder 28ʹ.

Further, the user may use the tool 250 to engage the set screw 214 to drive or compress the contact 180 against the grounding assembly 184, such as generally in the direction of arrow 256. Thus, the INS 32 may be held within the holder 28' and a grounding contact may be made against the INS 32 after positioning within the holder 28’. As discussed above, the grounding lead 46 may be contacted to the patient 42 and/or ensure its contact or connection with the grounding plug 120 to ground the INS 32 to the subject 42.

After positioning the INS 32 within the holder 28', the holder 28' with the INS 32 may be positioned in the pouch 24. It is understood, however, that the holder 28' may be assembled with the INS 32 at any appropriate time, such as assembly or disassembly may occur when the holder 28' is within the pouch 24. Nevertheless, the INS 32 may be an implantable device that is approved and/or trialed for approval with the subject 42 and/or a population rather than a separate trialing assembly.

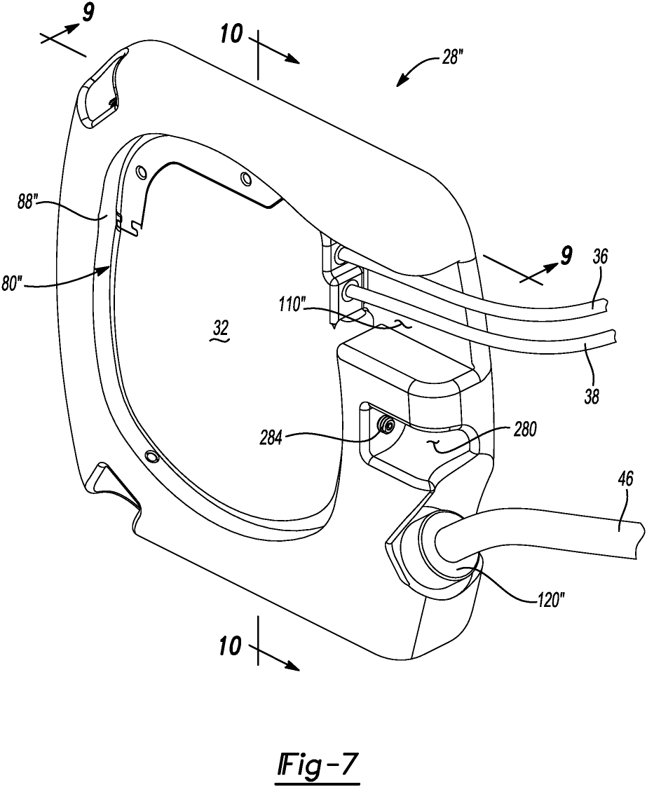

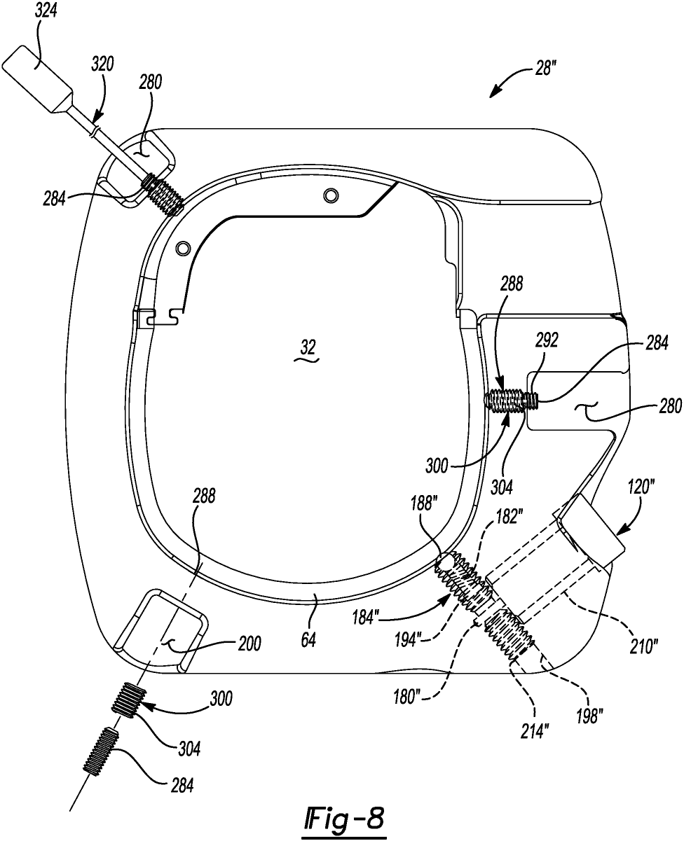

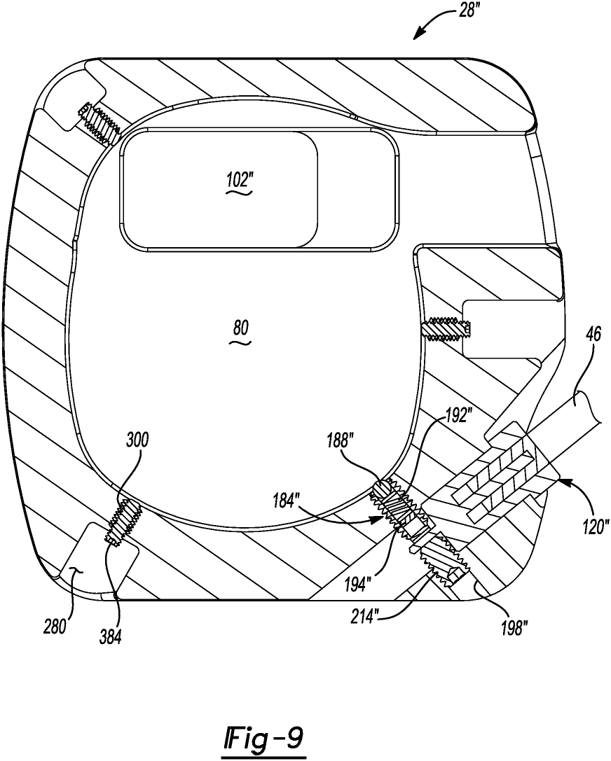

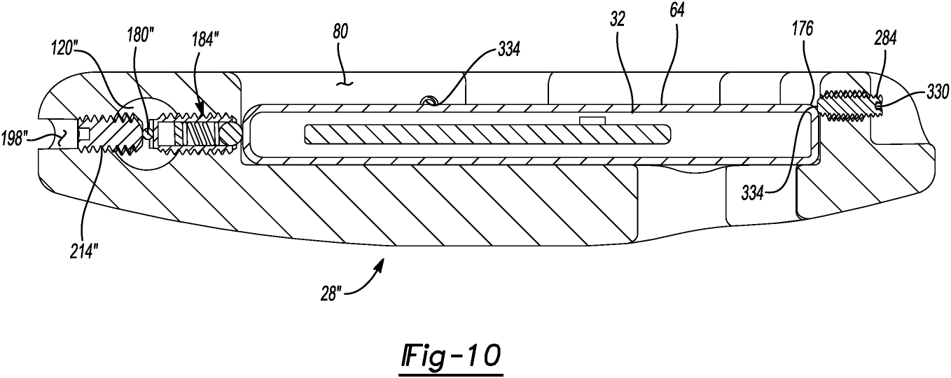

Turning reference to FIG. 7, FIG. 8, FIG. 9, and FIG. 10 a holder assembly 28ʺ is illustrated. The holder assembly 28ʺ may be substantially similar to the holder 28ʹ, as discussed above, and various similar features may include like or identical reference numerals augmented with a double prime (ʺ). It will be further understood that various features of the holder 28ʹ may be exchanged with the features of the holder 28ʺ and that they are not mutually exclusive unless specifically indicated herein. Accordingly, the holder 28ʺ may include a depression 80ʺ to engage or receive the INS 32. One or more internal walls 88ʺ may form the depression 80ʺ. The holder 28ʺ may include a passage 110ʺ to allow passage or access of the leads 36, 38 to the INS 32 positioned within the holder 28ʺ. A grounding port or member 120ʺ may be engaged or included with the holder 28ʺ to connect with the grounding lead 46. Thus, the holder 28ʺ may include portions to hold the INS 32 within the holder 28ʺ and allow for access or connection of the leads 36, 38 and grounding of the INS 32, similar to that discussed above.

Further, the holder 28ʺ may be formed of material similar to those discussed above regarding the holder 28ʹ. Likewise, similar formation processes, such as injection molding or machining, may be used to form the holder 28ʺ. The holder 28ʺ may also include appropriate geometry and dimensions to hold the INS 32 and be positioned with the pouch 24 relative to the subject 42. Thus, the holder 28ʺ may include portions or features that are substantially similar to the holder 28' as discussed above.

The holder 28ʺ, however, may include a plurality of holder portals 280 that allow access for positioning one or more holding set screws 284 positioned within holding passages 288. Each of the holding set screws 284 may engage the holder 28ʺ directly and/or indirectly such as with an external thread 292 of the set screw 284. The set screw 284 may be formed as a member that is passed into the passage 288 to hold or lock the INS 32 within the holder 28.

In various embodiments, for example, the holding passage 288 may be formed of a material that is tapped to engage the external threads 292 of the set screw 284. In various embodiments, however, the material of the holder 28ʺ may be soft enough or malleable enough to be tapped by the set screw 284 during insertion or positioning of the set screw 284. A still further embodiment, including in addition and/or alternatively to the above, an insert 300 may be positioned in the passage 288 that includes an external thread or surface 304 that engages the passage 288. The set screw 284 may then engage an internal surface or wall of the insert 300 to increase the holding power and/or fixing power of the set screw 284 relative to the holder 28ʺ.

As illustrated in the various figures, a plurality of the set screw 284 may be provided at various locations relative to the INS 32 to assist in holding the INS 32 within the holder 28ʺ, as discussed further herein. The set screw 284 may be formed of selected appropriate materials, such as stainless steel, other metal or metal alloys, appropriate polymers, or the like. For example, similar to the holding ball 150, the set screw 284 may be positioned to engaged an exterior or exposed surface of the INS 32 when the INS 32 is within the holder 28ʺ. Regardless, the set screw 284 may be used to assist in holding the INS 32 relative to the holder 28ʺ, as discussed further herein.

Each of the passages 280 allow for a tool, such as a tool 320 to engage the set screw 284 to torque the set screw 284 to a selected torque. For example, the set screws may be torqued to at least about 0.01 Newton-meters (n-m) (about 2 inch ounces (in-oz). As a further example, the torque may be about 0.03 n-m (about 5 in-oz) with a tolerance of 0.003 n-m (0.5 in-oz). Other appropriate torque amounts may be used, however, such as about 0.1 n-m (2 in-oz) to about 0.2 n-m (30 in-oz).

Accordingly, the tool 320 may include a torque handle or connection 324 that may be set to a selected torque, such as permanently set, to apply a selected torque to the set screw 284 to ensure appropriate holding power of the INS 32 within the holder 28”. Each of the passages 280 may be large enough to allow passage of the tool 320 and access the set screws 284. The material of the holder 28ʺ may include a selected toughness to allow the selected torque and/or the insert 300 may assist in ensuring an appropriate amount of torque may be applied to the set screw 284 against the INS 32.

With reference to FIG. 10, as discussed above, the set screw 284 may include a first end 330 that may be engaged by the tool 320 and a second or INS contacting end 334. As illustrated in FIG. 10, the contacting end 334 may engage the upper surface or surface 166 of the INS 32 and/or an upper or outer radius 176. In this manner, the holding or set screws 284 may hold the INS 32 from moving away or out of the depression 80 of the holder 28ʺ. The INS 32, therefore, may be held within the holder 28ʺ in an appropriate manner.

Further, as discussed above, the holder 28ʺ includes the grounding port or portion 120ʺ. The grounding portion 120ʺ may be held within a grounding passage 210ʺ. Further, the grounding portion 210ʺ includes the contact 180ʺ. The contact may be held against a grounding assembly 184ʺ similar or identical to the grounding assembly discussed above. Accordingly, briefly, the grounding assembly 184ʺ includes the grounding contact or ball 188ʺ that may contact the case 64. Further, the grounding assembly 184ʺ may include the biasing member 192ʺ that may also be electrically conductive and a holding portion 194ʺ. Thus, the grounding assembly 184ʺ may be substantially similar to the grounding assembly 184, as discussed above. Further, the contact 180ʺ of the grounding plug or contact 120ʺ may be engaged or held against the grounding assembly 184ʺ, similarly to that discussed above. A set screw 214ʺ may be positioned within a grounding passage 198ʺ and positioned against and compressed against the contact 180ʺ, as discussed above. Therefore, the grounding plug 120ʺ may be compressed against the grounding assembly 184ʺ to allow the grounding assembly 184ʺ to be in contact with the case 64 and have electrical continuity with the grounding plug 120ʺ. Accordingly, as discussed above, the grounding lead 46 may engage the grounding plug 120ʺ to ensure grounding with the casing 64 of the INS 32. Thus, the grounding assembly 184ʺ may have electrical continuity and contact between the INS 32 and the grounding port 120ʺ.

Additionally, similar to the holder 28', as discussed above, the holder 28ʺ may include a passage 102ʺ to assist in a user removing the INS 32 from the holder 28ʺ. The user may disengage or loosen the set screws 284 to reduce a holding force on the INS 32. The user may then extend or pass a member through the passage 102ʺ to assist in disengaging or removing the INS 32 from the holder 28ʺ.

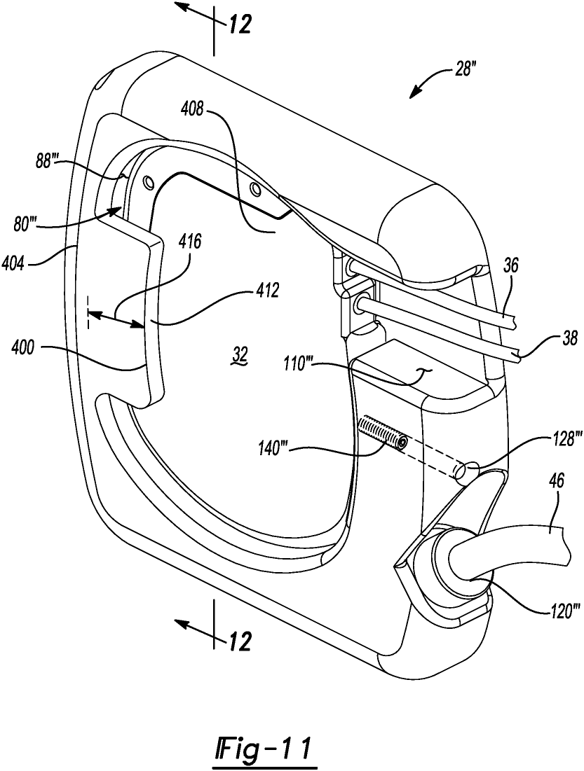

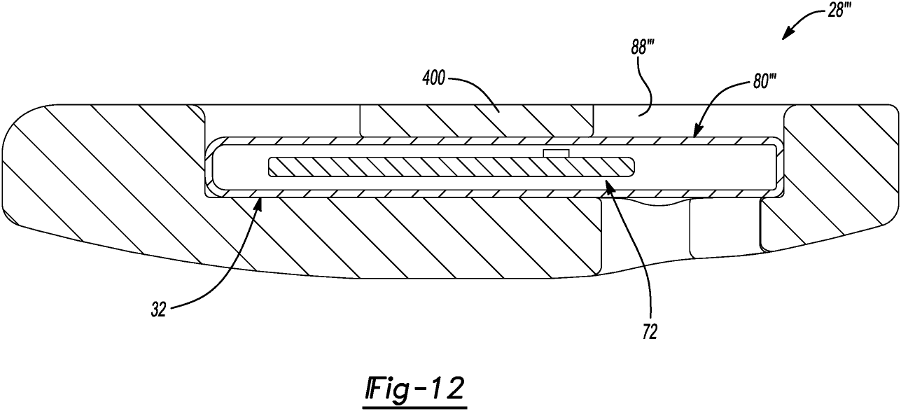

Turning reference to FIG. 11 and FIG. 12, a holder 28ʹʹʹ is illustrated. The holder 28ʺʹ may be similar to the holders discussed above, such as the holder 28' and the holder 28ʺ. Accordingly, like referencing numerals may be used to reference and identify similar or identical features and differences are discussed further herein.

The older 28ʺ may include various holding portions, such as the biasing holding assembly 140, as discussed above. The holding portions may be passed through holding passages 128ʺʹ as also discussed above. Further, the holder 28ʺʹ may include a passage 110ʺʹ to allow passage or access for the leads 36, 38. The INS 32, therefore, may be positioned within a depression 80ʺʹ of the holder 28ʺʹ The holder 28ʺʹ may further include the grounding plug or connection 120ʺʹ o allow access or connection for the grounding lead 46. Accordingly, the holder 28ʺʹ may hold the INS 32 for various trialing purposes and periods as discussed above. The INS 32, however, may be held within the depression 80ʺʹ with features of the holder 28ʺʹ.

For example, as illustrated in FIG. 11, a projection or holding finger 400 may extend from an upper surface 404 of the holder 28ʺʹ and over at least a portion of the depression 80ʺʹ to at least partially enclose or cover the depression. The holder or finger 400, therefore, may extend over a portion of the INS 32 and/or engage a portion of the INS 32, such as a selected surface 408. The finger or holding portion 400 may extend to an edge 412 and, therefore, extend a selected distance 416 from the internal wall 88ʺʹ to engage or assist in holding the INS 32 within the depression 80ʺʹ. The holding portion 400 may be substantially rigid to hold or resist removal of the INS 32 to hold the INS 32 within the holder 28ʺʹ.

With additional reference to FIG. 12, the INS 32 may be held within the holder 28ʺʹ with the projection or holding finger 400. The INS 32, therefore, may be engaged by a portion of the holder 28ʺʹ In various embodiments, for example, the holder 28ʺʹ may be molded or machined from a single material or single block such that the projection 400 is formed integrally or is a single piece with other portions of the holder 28ʺʹ. In various embodiments, for example, however, the projection or holder finger 400 may be removably engaged with the holder 28ʺʹ to assist in engaging and holding the INS 32 within the depression 80ʺʹ.

Therefore, the holder 28ʺʹ may include various integral features, such as ones formed as a single piece with the holder 28ʺʹ, to assist in holding an INS 32 within the depression 80ʺʹ. The holder 28ʺʹ may further include various INS engaging portions, such as the holding assembly 140ʹʹʹ to further assist in holding the INS 32 within the holder 28ʹʹʹ. In this manner, various features may be provided to engage the INS 32 within the holder 28ʹʹʹ to hold the INS 32 within the holder 28ʺʹ for the selected trialing period.

In light of the above, the holder 28, according to various embodiments, is provided to hold the INS 32 for a selected trialing period. The INS 32 may be identical and/or the same INS to be implanted in the subject 42 after a trialing period. Therefore, the INS 32 need not be redesigned from a temporary or trialing assembly after the trialing but prior to implantation. Thus, the time lapse between a trial and an implantation may be reduced and/or minimized. Moreover, the elimination of a trialing assembly may be achieved by using the INS 32 with the holder 28, according to various embodiments. Moreover, it is understood that the holder 28, according to various embodiments, may include features that are exchanged amongst the embodiments illustrated above. Moreover, a fixed or solid set screw assembly may be used to provide an electrical connection between the base 62 and the grounding lead 46, such as through the grounding port 120. Thus the grounding assembly 184, according to various embodiments, need not include a deformable or movable ball or member. Nevertheless, the holder 28, according to various embodiments, is configured to hold the INS 32 for a selected period.

As discussed above, the system 20 may include a holder, for example, the external holder 28, and it may be positioned relative to the subject 42 in an appropriate manner. In various embodiments, for example, the external pouch 24 may be used to hold the external holder 28 relative to the subject 42. The external holder 28 may be provided according to various embodiments, including those discussed above. For example, the external holder 28 may include that illustrated in FIG. 1 and be used to hold a selected device such as the implantable nerve stimulation system (INS) 32, as illustrated in FIG. 3. The external holder 28, however, may also be provided in various embodiments including that illustrated in FIGS. 5, 8, and 11.

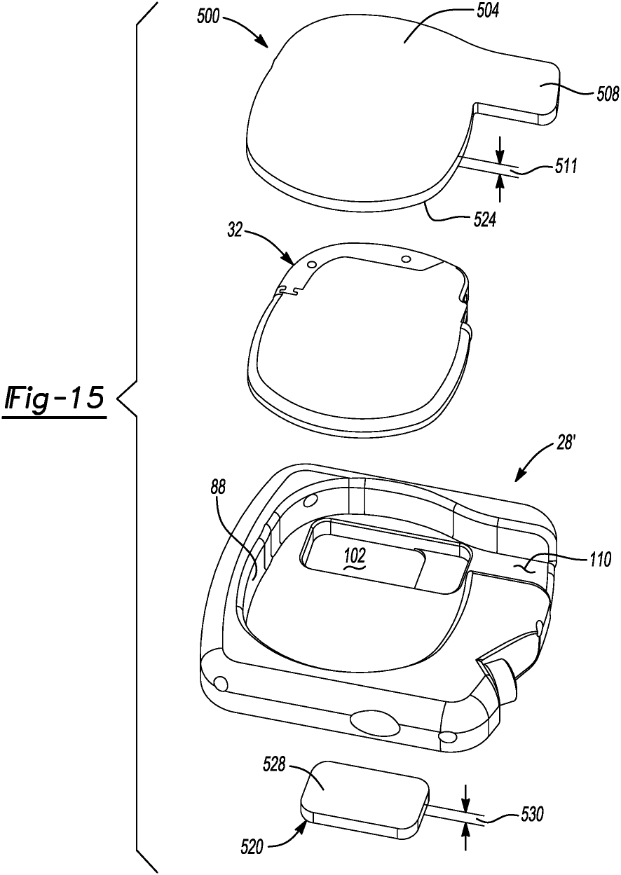

With reference to FIGS. 1-12, discussed above, and additional reference to FIGS. 13, 14, and 15, according to various embodiments, one or more covers may be provided with the external holders, according to various embodiments. For example, as illustrated in FIG. 13, the holder 28ʹ may include an opening or void that may be surrounded by the internal walls 88 in which the INS 32 may be positioned. A cover 500, also referred to as a first cover 500, may be positioned relative to, such as within, the internal walls 88 to cover at least a portion of the INS 32. As illustrated in FIGS. 13 and 15, the cover 500 may include a main portion or body 504 to cover the INS 32 and the region within the external holder 28ʹ holding the INS 32 and a tab or extension 508 to cover the side port 110. The cover 500 may cover a first side of the holder 28ʹ. The cover 500 may have any appropriate thickness 511, such as about 0.01 millimeters (mm) to about 1 centimeter (cm), including about 1 mm to about 3 mm.

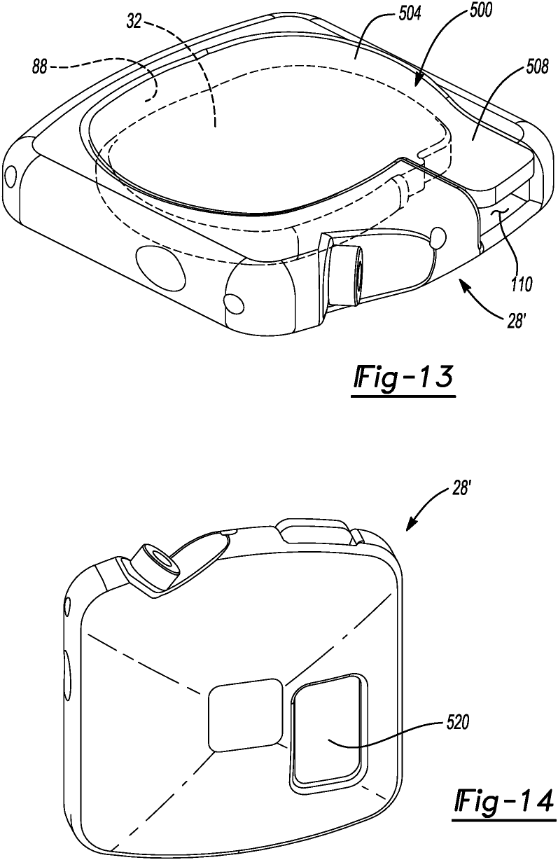

The holder 28ʹ may further include the passage or port 102, as illustrated in FIG. 5, that may be covered by a second cover 520 as illustrated in FIGS. 14 and 15. The second cover 520 may be fit within the passage 102 to restrict or limit access to the INS 32 within the external holder 28', as illustrated in FIG. 14. The port 102 may be formed, as illustrated and discussed above, through a second side of the holder 28ʹ. Thus, the second cover 520 could cover the opening on the second side opposite the first side by the first cover 500. The second cover 520 may have any appropriate thickness 530, such as about 0.01 millimeters (mm) to about 1 centimeter (cm), including about 1 mm to about 3 mm.

Reference to first cover 500 and second cover 520 is only for clarity of the subject disclosure.

With continuing reference to FIGS. 13 and 14 and additional reference to FIG. 15, the INS 32 may be positioned within the opening defined by the internal side walls 88 of the holder 28’. Various connections may also be made to the INS 32. Connections may include those made through the portal 110 after delivery of the assembly of the holder 28’ and the INS 32 to a user, such as a surgeon.

The cover 500 may be positioned to cover the INS 32 when the INS 32 is placed in the holder 28’ or at any appropriate time thereafter. As illustrated in FIG. 13, the cover 500 may be placed to cover the INS 32 by moving the cover 500 toward the INS 32 and the holder 28’. The cover 500 may then be connected to the INS 32 and/or the holder 28ʹ.

On an INS contacting side 524 of the cover 500 may be a selected connection feature, such as an adhesive. The adhesive may be a pressure-sensitive adhesive that is positioned on the INS contacting side 524. It is understood, however, that any appropriate adhesive or connection may be used to connect to the cover 500 to the holder 28’ and/or the INS 32. Nevertheless, an adhesive, such as a pressure-sensitive adhesive, may allow for a connection of the cover 500 to the INS 32 during use and application of the INS 32.

Further the cover 520 may be positioned relative o the holder 28’ at any appropriate time. The cover 520 may also have an INS contacting side 528. The INS contacting side 528 may also include a selected connection, such as an adhesive. The adhesive on the INS contacting side 528 may also include a pressure-sensitive adhesive similar or identical to the adhesive of the cover 500.

The cover 520 may connected to the holder 28’ and/or the INS 32. In various embodiments, the cover 520 may at least partially pass through the passage 102 and be adhered to the INS 32 in the holder 28ʹ. This allows the cover 520 to be fit within the passage 102.

The covers 500, 520 may be connected, such as adhered, to the INS 32 and/or the holder 28ʹ to cover the passage 102 and the opening over the INS 32 in the holder 28ʹ. The covers 500, 520, when positioned to cover the INS 32, may be generally flush with a surface of the holder 28ʹ. Thus, the covers 5200, 520 may not protrude from the holder 28ʹ

The covers 500, 520 may be provided for various purposes, such as to protect the INS 32 during use, cover the INS 32 during use, and other appropriate purposes. In various embodiments, the covers 500, 520 may be formed of a closed cell foam to provide a barrier, such as a liquid barrier to the INS 32 within the holder 28ʹ. The covers 500, 520 may also be formed of any appropriate material such as a material that is opaque to provide secrecy to the INS 32. The covers 500, 520 may be made and/or formed of appropriate materials. For example, the covers 500, 520 may exemplarily be made of or formed from materials including Ethylene Propylene Terpolymer Rubber (EPDM), SBR Foam, and Neoprene foam, and Moleskin material such as those sold by Grainger, Inc.

Further the covers 500, 520 may assist in protecting the INS 32. As discussed above, the INS 32 may be positioned within the holder 28ʹ and/or connected thereto. The cover 500, 520 may assist in holding the INS 32 within the holder 28ʹ. Further the covers 500, 520 may be formed of material to assist in reducing impact to the INS 32, such as from a patient, items exterior to the patient, or the like.

The covers 500, 520 may be provided relative to the holder 28’ for various purposes. The covers 500, 520 may be assembled to the holder 28' either after the INS 32 is positioned in the holder 28ʹ and, at least partially, prior thereto. For example, the cover 520 may be positioned within the passage 120 and connected to the holder 28ʹ. Thus, the cover 520 may be connected to the holder 28ʹ prior to positioning the INS 32 within the holder 28ʹ.

According to the above, therefore, holders according to various embodiments may include various covering portions that are provided separate from the holder 28ʹ. In various embodiments, however, the covers may also be integrated into the holder 28ʹ or a holder according to any embodiment to assist in covering partially and/or entirely the INS 32. Further, while the covers 500, 520 are illustrated and described relative to the holder 28ʹ, it is understood by one skilled in the art that identical and/or similar covers may be provided relative to the holders according to various embodiments, and combinations thereof. The covers may be provided for various purposes such as covering the INS 32 and/or assisting and protecting the INS 32 from contact and/or exposure.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, graphic processing units (GPUs), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. In addition, the techniques could be fully implemented in one or more circuits or logic elements.

What is claimed is:

1. A system to hold a therapy device relative to a subject, comprising:
a holder having a depression to hold the therapy device within a holder wall;
a biasing system configured to engage the therapy device within the holder to provide a selected holding force of the therapy device within the holder; and
a grounding system having a member extending through the holder to engage a grounding lead;
wherein the holder is configured to be held relative to subject for a selected period of time.

2. The system of claim 1, further comprising:
a pouch configured to hold the holder relative to the subject.

3. The system of claim 1, wherein the grounding system further comprises:
a ground biasing assembly having
an electrically conductive member;
an electrically conducting biasing member configured to bias the electrically conductive member toward the depression;
a set screw configured to hold the member in electrical contact with at least a portion of the ground biasing assembly.

4. The system of claim 3, wherein the ground biasing assembly has a cylinder configured to hold the electrically conductive member and the electrically conducting biasing member and the member is held in electrical contact with the cylinder.

5. The system of claim 1, wherein the biasing system further comprises:
a holding member; and
a biasing member configured to bias the holding member toward the depression.

6. The system of claim 5, further comprising:
a stimulation system held within the depression at least in part by the biasing system.

7. The system of claim 1, further comprising:
a cover configured to cover at least a portion of the holder;
wherein the holder includes at least one passage to receive the therapy device;
wherein the cover is configured to cover at least a portion of the therapy device when received within the holder.

8. A system to hold a therapy device relative to a subject, comprising:
a holder having a depression to hold the therapy device within a holder wall;
a compression member configured to engage the therapy device within the holder to provide a selected holding force of the therapy device within the holder; and
a grounding system having a member extending through the holder to engage a grounding lead;
wherein the holder is configured to be held relative to subject for a selected period of time.

9. The system of claim 8, wherein the compression member further comprises:
a threaded member configured to be driven toward the depression;
wherein a passage is formed within a wall of the holder to allow access of a driver to drive the threaded member.

10. The system of claim 9, further comprising:
a holding reinforcing member to increase a holding power of the threaded member relative to the wall.

11. The system of claim 9, further comprising:
a stimulation system held within the depression at least in part by the biasing system.

12. The system of claim 8, further comprising:
a pouch configured to hold the holder relative to the subject.

13. The system of claim 8, wherein the grounding system further comprises:
a ground biasing assembly having
an electrically conductive member;
an electrically conducting biasing member configured to bias the electrically conductive member toward the depression;
a set screw configured to hold the member in electrical contact with at least a portion of the ground biasing assembly.

14. The system of claim 13, wherein the ground biasing assembly has a cylinder configured to hold the electrically conductive member and the electrically conducting biasing member and the member is held in electrical contact with the cylinder.

15. The system of claim 8, further comprising:
a cover configured to cover at least a portion of the depression of the holder;
wherein the depression is configured to receive the therapy device;
wherein the cover covers at least a portion of the therapy device when received within the depression.

16. A system to hold a therapy device relative to a subject, comprising:
a holder having (i) a depression adapted to receive the therapy device within a holder wall and (ii) a projection extending from the holder wall to at least partially enclose the depression;
a holding member configured to engage the therapy device within the holder to provide a selected holding force of the therapy device within the holder; and
a grounding system having a member extending through the holder to engage a grounding lead;
wherein the holder is configured to be held relative to subject for a selected period of time.

17. The system of claim 16, wherein the holding member comprises at least one of (1) a threaded member configured to be driven toward the depression, (2) biasing system, or (3) combinations thereof.

18. The system of claim 17, wherein the biasing system further comprises a holding member and a biasing member configured to bias the holding member toward the depression.

19. The system of claim 17, further comprising:
a stimulation system held within the depression at least in part by the biasing system.

20. The system of claim 16, wherein the grounding system further comprises:
a ground biasing assembly having
an electrically conductive member;
an electrically conducting biasing member configured to bias the electrically conductive member toward the depression;
a set screw configured to hold the member in electrical contact with at least a portion of the ground biasing assembly.

21. The system of claim 20, wherein the ground biasing assembly has a cylinder configured to hold the electrically conductive member and the electrically conducting biasing member and the member is held in electrical contact with the cylinder.

22. The system of claim 20, further comprising:
a cover configured to cover at least a portion of the depression of the holder;
wherein the cover covers at least a portion of the therapy device when received within the depression.
